(19) United States
(12) Patent Application Publication
(10) Pub. No.: US 2024/0043471 A1
(43) Pub. Date: Feb. 8, 2024

(54) PREPARING METHOD OF HIGHLY FUNCTIONAL PEPTIDE DERIVED FROM KERATINOCYTE PROTEIN

(71) Applicant: ANPEP INC., Cheongju-si (KR)

(72) Inventors: Sang Moon KANG, Daejeon (KR); Kae Young LEE, Cheongju-si (KR); Kwang Soon CHOI, Cheongju-si (KR); Sang Joo CHA, Goyang-si (KR)

(73) Assignee: ANPEP INC., Cheongju-si (KR)

(21) Appl. No.: 18/348,878

(22) Filed: Jul. 7, 2023

Related U.S. Application Data
(63) Continuation of application No. 17/546,739, filed on Dec. 9, 2021.

(51) Int. Cl. C07K 5/103; A61Q 19/08; A61K 8/64

(52) U.S. Cl. CPC C07K 5/1008; A61Q 19/08; A61K 8/64

(57) ABSTRACT

There is provided a method of synthesizing the tetrapeptide exhibiting an anti-wrinkle activity consisting of the amino acid sequence of glycine(Gly)-glutamine(Gln)-valine(Val)-serine(Ser) (SEQ ID NO: 1). The method includes: preparing a peptidyl resin by utilizing 2-(4-nitrophenyl) sulfonylethoxycarbonyl-amino acid (Nsc-amino acid); adding an eluting solution to the prepared resin; and filtering the resin and obtaining the tetrapeptide.

### CROSS-REFERENCE TO PRIOR APPLICATION

This Application is a Continuation Application of U.S. patent application Ser. No. 17/546, 739 (filed on Dec. 9, 2021), which is hereby incorporated by reference in its entirety.

### SEQUENCE LISTING

This Application contains a Sequence Listing submitted via XML file and hereby incorporated by reference in its entirety. The Sequence Listing is named “659-0038A_Sequence_Listing.xml”, created on Jun. 19, 2023 and 52,626 bytes in size.

### BACKGROUND

The present invention relates to a highly functional peptide, and more specifically, to a highly functional peptide derived from a natural fermentation product.

The present invention is derived from research conducted with research funds supported by core technology development project for bio-industry based on Purchase Conditions of the Ministry of Trade, Industry and Energy.

[Project unique number: 1415168661, Name of research project: Development of a novel from of dermacosmetic peptidomimetics by In silico virtual screening]

The skin is the largest tissue in the human body. It functions to protect the inside of the body from sunlight or physical and chemical stimuli, is absolutely necessary for human life, and is constantly regenerated to maintain homeostasis. The skin is composed of the epidermis, dermis, and subcutaneous fat from the outside in this order. The epidermis, which is the thinnest tissue, has an important role of moisturizing and protecting the skin, and has a role of preventing moisture loss, damage, invasion of bacteria, etc. The skin is a tissue which covers the entire body and has various functions. Since it has a barrier function between the inside and outside of the body, it has an immune system by islets of Langerhans and has a moisture control function by the epidermis. It also synthesizes vitamin D with adequate sunlight and performs the functions of lipid storage and excretion into sweat glands. The stratum corneum produced by the normal differentiation process in healthy skin has the function of maintaining skin moisture and protecting it from external environmental stimuli. Such a function is called a skin barrier function and can be regarded as the most important role of the epidermis.

One of the biggest areas of interest in cosmetics is senescence associated with aging, pigmentation, loss of elasticity, xeroderma, hair loss, lack of luster of hair, etc. The skin undergoes various changes through aging. First, the thickness of the epidermis, dermis, and subcutaneous tissue, which are components of the skin, becomes thinner, and the extracellular matrix (ECM) components that give elasticity to the skin change. Among them, collagen, which accounts for 70-80% of the extracellular matrix, undergoes a decrease in its production with age and has a close relationship with the generation of aging, while collagen, elastin, proteoglycans, glucosaminoglycan, laminin, fibronectin, etc. that make up the skin's connective tissue are oxidized and lose their functions to thus allow the skin to lose its elasticity and aging to be formed excessively, thereby being transformed into senile skin.

The stratum corneum protects our body from the external environment and functions as the frontmost barrier that prevents the body fluid from being lost to the outside. During differentiation of keratinocytes, their cell membranes are replaced by a unique structure called the “cornified envelope”. In the keratinocyte membrane, several insoluble proteins are cross-linked to form a structure, which forms a physical barrier by forming a covalent bond with some lipid components constituting the lipid membrane to protect the human body from external threats in a state being surrounded by a lipid membrane outside the cell. In particular, various structural proteins present in keratinous tissue are known to play an important role in the formation of epidermal tissue and a barrier function, and there are various skin diseases caused by the absence of structural proteins in keratinocytes.

Connective tissue fibers of the extracellular matrix include collagen fibers, reticular fibers, and elastic fibers. Among them, collagen which accounts for about 70% of the skin connective tissue is mostly formed in the fibroblasts of the skin. Collagen content in the skin connective tissue decreases with age, which is due to a decrease in collagen synthesis and promotion of its decomposition. Therefore, the decrease in collagen biosynthesis and the promotion of collagen degradation are the biggest causes of skin aging.

The collagen biosynthesis process is regulated by many factors involved in the transcription level and post-translation level and causes changes. Collagen decomposition is stimulated by the expression of matrix metalloproteases (MMPs) (e.g., collagenase), which decomposes collagen by ultraviolet rays, etc. thereby reducing the collagen content. In addition, as the modification of collagen is accelerated by the external environment, the skin aging progresses frequently and further. Consequently, the skin aging phenomenon is caused by non-uniformity of cells, loss of elastin, destruction of collagen, decrease and delay of collagen synthesis, etc. Therefore, although skin aging also occurs in the epidermis, it can be seen as a phenomenon that occurs in the dermis rather than in the epidermis.

Studies on various cosmetic compositions are underway as a way to solve these skin aging-related problems, and the skin aging effect is showing tangible results in some cases. For example, various clinical results of retinoids (especially retinol among them), which are widely used for the improvement of skin aging, melasma, and pigmentation improvement, with regard to elimination of skin aging have been reported variously, and cosmetics containing retinol showed effective improvements on aged skin, skin sagging, loss of elasticity generated by sunlight, etc.

In recent years, with the increase of human lifespan and the improvement of standard of living, people's interest on health and beauty has been greatly increased. This can be clearly seen from the recent rapid increase in the markets for functional cosmetics, functional foods, and beauty industry related to skin aging.

There is a relatively large technology gap between Korea and advanced countries in skin aging-related technologies, and most of the research on aging being led by the Korean Society for Gerontology, is focused on the inhibition of antioxidant activity as basic research such as aging mechanism, etc. The overall level of competitiveness in the field of anti-aging science is at the level of about 75% compared to that of the world's best technology-holding country (USA), and the technology gap was shown to be 4.5 years. The technical field where Korea has the highest level of technological competitiveness is the e field of technology development relating to aging control, which is at the level of 74.5% of the world's best technology holding countries. The fields of aging research in Korea are very diverse, and among them, the research on the fields of cranial nerve, blood vessels, joints, recognition and perception, cancer, diabetes, and obesity are actively underway. However, compared to other research fields, research on skin aging has an insufficient interest, investment, and studies, and there is a need for improvement due to the absence of systematic and integrated research from the viewpoint of aging.

Recently, with the extension of the use of anti-aging products to young women in their late 20s, these anti-aging products are actively developed and released. In particular, in order to meet the various needs of consumers in the cosmetic industry, the scope of these products is not limited to the cosmetic field, but is being expanded to nutricosmetics, nutritional supplements, esthetics, high-functional cosmetics, cosmetic procedures, etc. Currently, a wide range of research is being conducted on the subject of aging, spanning from the research on the aging mechanism to policy research (e.g., support for policy development relating to society, medicine, welfare for the elderly, etc.). In particular, much manpower and money are being invested in the development and commercialization of anti-aging technology relating to fermentation.

For effective prevention or improvement of skin aging and senescence, there is a need for the development of a novel anti-aging composition using raw materials whose efficacies have been accurately confirmed, and it is important to confirm these efficacies by way of identifying molecular biological mechanisms and through human clinical trials.

### SUMMARY

Accordingly, the present invention provides a novel peptide, which is a highly functional peptide derived from a natural fermentation product, exhibiting an activity of inhibiting wrinkles on the skin and an anti-wrinkle cosmetic composition containing the same.

In order to solve the problems above, the present invention provides a tetrapeptide exhibiting an anti-wrinkle activity consisting of an amino acid sequence of glycine (Gly)-glutamine(Gln)-valine(Val)-serine(Ser) (SEQ ID NO: 1).

Additionally, the present invention provides a tetrapeptide exhibiting an anti-wrinkle activity consisting of an amino acid sequence of glycine (Gly)-glutamine(Gln)-aspartic acid(Asp)-proline(Pro) (SEQ ID NO: 2).

Additionally, the present invention provides a tetrapeptide, in which the peptide is synthesized by utilizing 2-(4-nitrophenyl)sulfonylethoxycarbonyl-amino acid (Nsc-amino acid) as an intermediate.

Additionally, the present invention provides a tetrapeptide, in which the anti-wrinkle activity is exhibited through the inhibition of elastase.

Additionally, the present invention provides a tetrapeptide, in which the anti-wrinkle activity is exhibited through collagen synthesis.

In order to solve another object, the present invention provides an anti-wrinkle cosmetic composition containing the peptide.

The tetrapeptide according to the present invention exhibits an anti-wrinkle activity through inhibition of elastase or collagen synthesis, and thus can have various utilities as a cosmetic composition.

### BRIEF DESCRIPTION OF THE DRAWINGS

FIG. 1 is a flowchart illustrating the overall peptide synthesis process in Example 1 of the present invention.

FIG. 2 is a graph illustrating the measurement results of collagen production in Example 3 of the present invention.

FIGS. 3 to 5 are graphs illustrating the experimental results of the cell proliferation ability in Example 4 of the present invention.

FIGS. 6 and 7 are graphs illustrating the HPLC analysis results of the tetrapeptides of Sample 10 and Sample 34 in Example 5 of the present invention, respectively.

FIGS. 8 and 9 are graphs illustrating the experimental results of thermal stability and photostability in Example 6 of the present invention.

### DETAILED DESCRIPTION

Hereinafter, the present invention will be described in detail through preferable embodiments. First of all, it will be understood that words or terms used in the specification and claims shall not be interpreted as the meaning defined in commonly used dictionaries. It will be further understood that the words or terms should be interpreted as having a meaning that is consistent with their meaning in the context of the relevant art and the technical idea of the invention, based on the principle that an inventor may properly define the meaning of the words or terms to best explain the invention. Therefore, the features of the embodiments and drawings described herein are merely the most preferable exemplary embodiment for the purpose of illustrations only, not intended to represent all the technical concepts of the disclosure, and thus it should be understood that various modifications and equivalents could be made thereto at the time of present application. Furthermore, in the entire specification, when it is described that one part “includes” some components, it does not mean that other components are excluded but means that other elements may be further included if there is no specific contrary description.

The present inventors have found that a tetrapeptide with a specific amino acid sequence derived from a fermented product of a ginseng fruit exhibits an excellent anti-wrinkle activity through inhibition of elastase or collagen production, thereby leading to the present invention.

Accordingly, the present invention discloses a tetrapeptide exhibiting an anti-wrinkle activity consisting of an amino acid sequence of glycine (Gly)-glutamine(Gln)-valine(Val)-serine(Ser) (SEQ ID NO: 1) and a tetrapeptide exhibiting an anti-wrinkle activity consisting of an amino acid sequence of glycine (Gly)-glutamine(Gln)-aspartic acid(Asp)-proline(Pro) (SEQ ID NO: 2).

In another aspect, the present invention provides an anti-wrinkle cosmetic composition containing the tetrapeptide above.

The tetrapeptide may be contained in an amount of 0.001 wt % to 10 wt %, preferably 0.01 wt % to 5 wt %, and more preferably 0.05 wt % to 1 wt %, based on the total cosmetic composition.

The cosmetic composition according to the present invention may be applied to a gel type, skin type, cream type, ointment type, etc., but is not limited thereto. The composition may be appropriately prepared by a known method by adding an appropriate conventional softening agent, emulsifying agent, thickening agent, or other materials known in the art according to their type.

The gel-type composition may be prepared by adding an emollient (e.g., trimethylolpropane, polyethylene glycol, glycerin, etc.), a solvent (e.g., propylene glycol, ethanol, and isostatic alcohol, etc.), purified water, etc.

The skin-type composition may be prepared by adding fatty alcohols (e.g., stearyl alcohol, myristyl alcohol, behenyl alcohol, arachidyl alcohol, isostearyl alcohol, isocetyl alcohol, etc.), butylene glycol, glycerin, allantoin, methylparaben, EDTA-2-sodium, xanthan gum, dimethicone, polyethylene glycol-60 hydrogenated castor oil, polysorbate 60 and, purified water, etc.

The cream-type composition may be prepared by adding fatty alcohols (e.g., stearyl alcohol, myristyl alcohol, behenyl alcohol, arachidyl alcohol, isostearyl alcohol, isocetyl alcohol, etc.), lipids (e.g., lecithin, phosphatidylcholine, phosphatidylethanolamine, phosphatidylserine, phosphatidylinositol, and derivatives thereof; emulsifying agents (e.g., glyceryl stearate, sorbitan palmitate, sorbitan stearate, etc.); natural fats or oils (e.g., avocado oil, apricot oil, babassu oil, borage oil, camellia oil, etc.); solvents (e.g., propylene glycol, etc.); purified water, etc.

The ointment-type composition may be prepared by adding an emollient, an emulsifying agent, and a wax (e.g., microcrystalline lead, paraffin, ceresin, beeswax, spermaceti, vaseline, etc.).

The cosmetic composition of the present invention may further contain one or wrinkle-improving ingredients more exhibiting the same or similar function in addition to the active ingredients. The wrinkle-improving ingredient may be any one or more selected from the group consisting of vitamin C, retinoic acid, TGF, animal placenta-derived proteins, betulinic acid, and a chlorella extract, but is not limited thereto. In addition, the cosmetic composition of the present invention may further contain excipients including a fluorescent material, a fungicide, a hydrotrope, a humectant, a fragrance, a fragrance carrier, a protein, a solubilizer, a sugar derivative, a sun blocking agent, vitamins, plant extracts, etc.

In the present invention, the cosmetic composition may be formulated into an emollient lotion, an astringent lotion, a nourishing lotion, an eye cream, a serum, a nourishing cream, a massage cream, a cleansing cream, a cleansing lotion, a cleansing foam, a cleansing water, a powder, an essence, a pack, a hair tonic, a hair treatment, a shampoo, or a conditioner.

The cosmetic composition may be formulated by a conventional method. With regard to the formulation of an external skin preparation, reference may be made to the contents disclosed in Remington's Pharmaceutical Science, Mack Publishing Company, Easton PA; and with regard to the formulation of a cosmetic composition, reference may be made to the contents disclosed in the International cosmetic ingredient dictionary, 6th ed. (The cosmetic, Toiletry and Fragrance Association, Inc., Washington, 1995).

Specifically, the cosmetic composition may be prepared into general emulsified formulations and solubilized formulations. For example, the cosmetic composition may be prepared into a lotion (e.g., an emollient lotion or a nourishing lotion); an emulsion (e.g., a facial lotion, a body lotions, etc.); a cream (e.g., a nourishing cream, a moisturizing cream, an eye cream, etc.); an essence; a makeup ointment; a spray; a gel; a pack; a sun blocking agent; a makeup base; a foundation (e.g., a liquid type, a solid type, a spray type, etc.); a powder; a makeup remover (e.g., a cleansing cream, a cleansing lotion, a cleansing oil, etc.); or a cleansing agent (e.g., a cleansing foam, a soap, a body wash, etc.), but is not limited thereto. In addition, the external preparation for skin may be formulated into an ointment, a patch, a gel, a cream or spray, but is not limited thereto.

In the cosmetic composition, in addition to the essential ingredients in each formulation, other ingredients may appropriately be mixed within the range that does not impair the purpose of the present invention according to the type of formulation, purpose of its use, etc.

The cosmetic composition may include a conventionally acceptable carrier, for example, an oil fraction, water, a surfactant, a humectant, a lower alcohol, a thickener, a chelating agent, a colorant, a preservative, a fragrance, etc. may appropriately be mixed, but is not limited thereto no.

The acceptable carrier may vary depending on the formulation. For example, when the composition is formulated into an ointment, paste, cream, or gel, an animal oil, a vegetable oil, a wax, paraffin, starch, tragacanth, a cellulose derivative, polyethylene glycol, silicone, bentonite, silica, talc, zinc oxide, or a mixture thereof may be used.

When the cosmetic composition is formulated into a powder or spray, as a carrier component, lactose, talc, silica, aluminum hydroxide, calcium silicate, polyamide powder, or a mixture thereof may be used. In the case of a spray, a propellant (e.g., chlorofluorohydrocarbon, propane, butane, and dimethyl ether) may be further included.

When the cosmetic composition is formulated into a solution or emulsion, as a carrier component, a solvent, a solubilizing agent, or an emulsifying agent may be used. For example, water, ethanol, isopropanol, ethyl carbonate, ethyl acetate, benzyl benzoate, propylene glycol, and 1,3-butylglycol oil may be used; and in particular, cottonseed oil, peanut oil, corn germ oil, olive oil, castor oil and sesame oil, glycerol fatty esters, or fatty acid esters of polyethylene glycol or sorbitan may be used.

When the cosmetic composition is formulated into a suspension, as a carrier component, a liquid diluents (e.g., water, ethanol, and propylene glycol); a suspending agent (e.g., ethoxylated isostearyl alcohol, polyoxyethylene sorbitol esters, and polyoxyethylene sorbitan esters); microcrystalline cellulose; aluminum metahydroxide; bentonite; agar or tragacanth, etc. may be used.

The cosmetic composition, depending on the quality or function of the final product, may further include a fatty material, an organic solvent, a solubilizing agent, a thickening agent, a gelling agents, a softening agent, an antioxidant, a suspending agent, a stabilizing agent, a foaming agent, a fragrance, a surfactant, water, an ionic or non-ionic type emulsifying agent, a filler, a sequestering agent, a chelating agent, a preservative, a blocking agent, a humectant, an essential oil, a dye, a pigment, a hydrophilic or lipophilic activating agent commonly used in the industry; adjuvants commonly used in the field of cosmetology or dermatology, such as any other ingredients commonly used in cosmetics. However, the adjuvant and a mixing ratio thereof may appropriately be selected so as not to affect the desirable properties of the cosmetic composition according to the present invention.

Hereinafter, the present invention will be described by way of specific embodiments according to the present invention.

### Preparation Example: Preparation of Fermented Product of Ginseng Fruit

After adding purified water (2 kg) to dried ginseng fruit (200 g), hot water extraction was performed at 120° C. for 6 hours. After extraction, a ginseng fruit extract having a sugar content of 6 to 8 birx was used. After the extraction, a Lactobacillus paracasei strain (purchased from the Korean Cell Line Bank (KCLB)) was cultured and the liquid culture was inoculated into the ginseng fruit extract in an amount of 1 wt % at a concentration of 1.0×10⁹ cfu/mL (measured by a spectrophotometer), and allowed to ferment at 37° C. for 3 days. The medium used for the cultivation was a medium consisting of glucose (0.6 wt %), an enzyme extract (0.3 wt %), soytone (0.1 wt %), and distilled water. After the fermentation, the fermentation liquid was centrifuged at 4,000 rpm for 10 minutes to separate lactic acid bacteria and a culture thereof, and then the culture was filtered through a 0.2 μm filter to prepare a fermentation product of ginseng fruit.

### Example 1: Synthesis of Peptide Library

The fermentation product of ginseng fruit was purified and isolated, and a total of 42 peptide sequences were derived therefrom, and these peptide sequences are shown in Table 1 below.

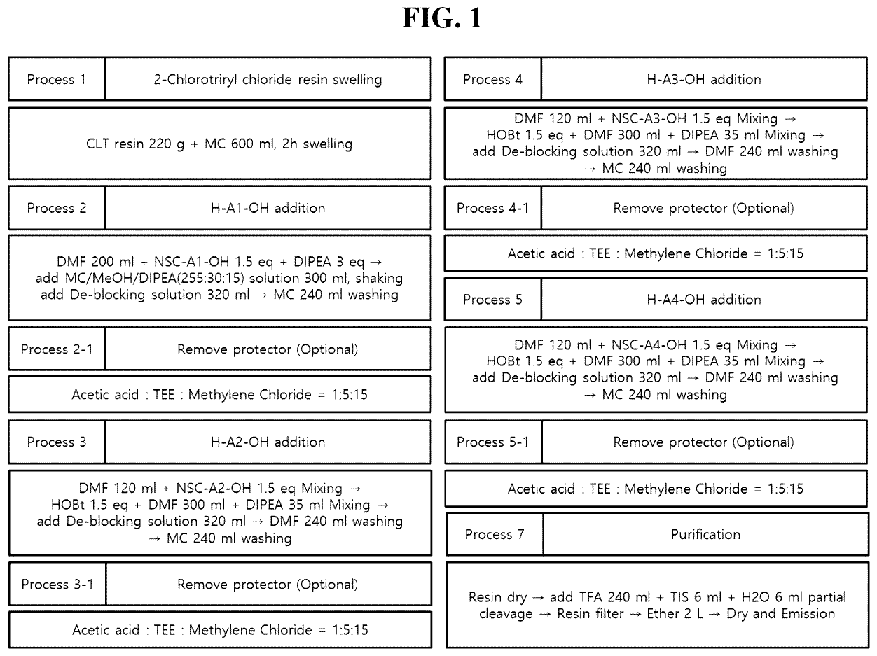

In order to construct a peptide library for synthesizing the peptides of Table 1, a chlorotrityl chloride resin (CTL resin, Novabiochem Cat. No. 01-64-0021), to which 19 nsc-amino acids (nsc-Ala, nsc-Arg (pbf), nsc-Asp (OtBu), nsc-Asn (trt), nsc-Gly, nsc-Glu (OtBu), nsc-Gln (trt), nsc-His (trt), nsc-Ser(tBu), nsc-Thr (tBu), nsc-Tyr (tBu), nsc-Trp (Boc), nsc-Leu, nsc-Ile, nsc-Val, nsc-Phe, nsc-Met, nsc-Lys (Boc), and nsc-Pro) are attached, was in series into a 96-well Teflon reactor in an amount of 50 mg for each of 19 lines, and methylene chloride (MC, 1 mL) was added thereto, stirred for 3 minutes. After removing the solution, 1 mL of dimethylformamide (DMF) was added thereto, and the mixture was stirred for 3 minutes, and the solvent was removed again. The prepared peptidyl resin was washed 3 times with DMF, MC, and methanol, respectively, dried by slowly flowing nitrogen air thereto, and then dried completely by reducing under vacuum in the presence of P₂O₅. After adding 30 mL of an eluting solution (containing trifluroacetic acid (TFA) (81.5%), distilled water (5%), thioanisole (5%), phenol (5%), 1, 2-ethanedithiol (EDT) (2.5%), and triisopropylsilane (TIS, 1%) to the prepared resin, and the reaction was maintained for one hour in an ice bath with occasional shaking at room temperature. The resin was filtered, washed with a small amount of TFA solution, and combined with the mother liquor. Thereafter, a tetrapeptide was obtained. The overall schematic diagram for the peptide synthesis is shown in FIG. 1.

As a result of the synthesis and purification above, 42 types of tetrapeptides having different sequences were obtained (Table 1). Although the yield of the synthesized tetrapeptides varied depending on the difference in physical properties due to the difference in the sequences of the peptides, the average yield was about 20%. From the column separation, it was confirmed that the elution time also varied, but the peaks were shown in the region of 6-8 minutes.

TABLE 1

| | | SEQ ID | | | SEQ ID |
|---|---|---|---|---|---|
| Sample | Sequence | NO | Sample | Sequence | NO |
| 1 | MSYQ | 3 | 22 | FSSG | 23 |
| 2 | QKKQ | 4 | 23 | GQLE | 24 |
| 3 | PTPQ | 5 | 24 | ELPE | 25 |
| 4 | PPVD | 6 | 25 | EQQE | 26 |
| 5 | DCVK | 7 | 26 | LELP | 27 |
| 6 | VKTS | 8 | 27 | GQLK | 28 |
| 7 | KTSG | 9 | 28 | KHLE | 29 |
| 8 | SGGS | 10 | 29 | EHQE | 30 |
| 9 | GSGY | 11 | 30 | EGQL | 31 |
| 10 | GQVS | 1 | 31 | LEVP | 32 |
| 11 | GYVS | 12 | 32 | PEEQ | 33 |
| 12 | SQQV | 13 | 33 | QMGQ | 34 |
| 13 | TQTS | 14 | 34 | GQDP | 2 |
| 14 | CAPQ | 15 | 35 | QLKY | 35 |
| 15 | QPSY | 16 | 36 | YLEQ | 36 |
| 16 | YGGG | 17 | 37 | EQQE | 37 |
| 17 | SSGG | 18 | 38 | TKGE | 38 |
| 18 | GGSG | 19 | 39 | VLLP | 39 |
| 19 | GSSG | 20 | 40 | VEHQ | 40 |
| 20 | GCFS | 21 | 41 | QQKQ | 41 |
| 21 | CFSS | 22 | 42 | EVQW | 42 |

### Example 2: Screening of Tetrapeptides

In order to confirm the anti-wrinkle effect of tetrapeptides, a test of inhibiting elastase was performed. The amount of p-nitroanilide produced was measured using N-succinyl-(L-Aal)3-p-nitroanilide as a substrate at 37° C. for 30 minutes. After adding each of the peptides to be tested and negative and positive controls in an amount of 100 μg/mL into test tube, respectively, a pancreatic solution (type I: derived from porcine pancreas, 0.6 unit/mL, Sigma Aldrich, USA) in an amount of 50 μg/mL was added thereto. Thereafter, a substrate, in which N-succinyl-(L-Ala)3-p-nitroanilide (1 mg/mL) was dissolved in 50 mM Tris-HCl buffer (pH 8.6), was added in an amount of 100 μg/mL thereto and allowed to react for 30 minutes and the absorbance was measured at 410 nm using a microplate reader. The elastase inhibitory activity was expressed as a decrease rate in absorbance of the groups with and without the addition of the sample solution as shown in Equation 1 below.

$$Elastase inhibition rate ( \% ) = \frac{absorbance of control - absorbance of sample}{absorbance of control} \times 100 \qquad [ Equation 1 ]$$

After treating the 42 kinds of synthesized peptides, their effects on inhibition of elastase were measured. The results are shown in Table 2 below.

TABLE 2

| | Elastase |
|---|---|
| | Inhibition |
| Sample | Rate(%) |
| 1 | 79.4 |
| 2 | 88.7 |
| 3 | 69.3 |
| 4 | 72.5 |
| 5 | 118.4 |
| 6 | 66.3 |
| 7 | 44.5 |
| 8 | 49.6 |
| 9 | 56.3 |
| 10 | 33.9 |
| 11 | 78.5 |
| 12 | 49.3 |
| 13 | 59.0 |
| 14 | 77.2 |
| 15 | 63.5 |
| 16 | 67.1 |
| 17 | 77.0 |
| 18 | 103.5 |
| 19 | 58.3 |
| 20 | 55.2 |
| 21 | 47.4 |
| Untreated | 100 |
| Group | |
| 22 | 69.1 |
| 23 | 73.2 |
| 24 | 90.5 |
| 25 | 53.6 |
| 26 | 78.2 |
| 27 | 56.0 |
| 28 | 79.3 |
| 29 | 66.3 |
| 30 | 69.7 |
| 31 | 70.6 |
| 32 | 64.0 |
| 33 | 55.6 |
| 34 | 34.5 |
| 35 | 60.5 |
| 36 | 72.3 |
| 37 | 64.2 |
| 38 | 59.3 |
| 39 | 60.7 |
| 40 | 55.7 |
| 41 | 72.3 |
| 42 | 49.5 |
| Positive | 28.8 |
| Group | |
| (Ursonic | |
| acid) | |

Referring to Table 2, it was found that the synthesized tetrapeptides having excellent elastase inhibitory ability (%) are even superior to that of ursonic acid, which is an existing functional material for inhibiting elastase. From this result, it can be seen that the synthesized tetrapeptides are effective in preventing skin aging by inhibiting wrinkle formation through their effects of inhibiting elastase when factors that induce wrinkle formation are applied to the skin.

Candidate peptides showing good titer by screening were classified into separate groups and synthesized on a large scale and were subjected to experiments.

### Example 3: Test of Intracellular Collagen Production Ability of Tetrapeptides

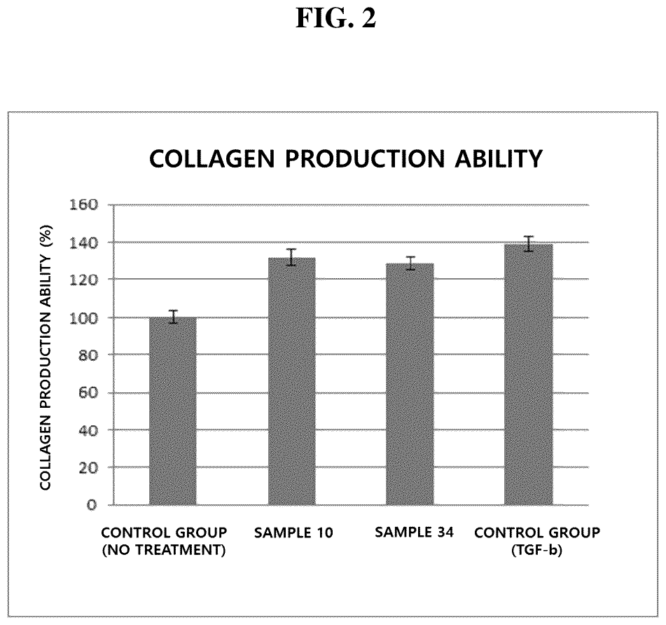

For the two types of tetrapeptides selected from the test of elastase inhibition ability (i.e., Sample 10 and Sample 34), their intracellular collagen production abilities were tested. The human dermal fibroblst (HDF) cells to be used in the test were cultured in a medium, in which 1× Low Serum Growth Supplement (Cascade Biologics™, S-003-10) was added to Medium 106 (Cascade Biologics™, M-106-500), under the conditions of 37° C. and 5% CO₂. The cells were cultured in a 24-well plate at a concentration of 1×10⁵ cells/well and the cell adhesion was examined. For the control group, only a solvent was added without any treatment. The dishes were added 4 types of experimental groups and a control group, which were each treated with TGF to a concentration of 10 μg/mL. After adding the test material to each dish, it was cultured for 2 days. After 2 days, each medium was collected and collagen production therein was measured using a Procollagen Type I C-peptide (hereinafter “PIP”) EIA kit (Takara MK101). The results are shown in FIG. 2.

Referring to Table 2, it was found that as a result of measuring collagen production after treating the two experimental groups according to concentration, the amount of collagen production was insufficient in most of the experimental groups compared to the control group (i.e., TGF); however, the amount of collagen production was higher than that of the negative control group. From these results, it can be seen that the wrinkles on the skin can be inhibited by increasing collagen production according to the sequences of these tetrapeptides, as compared to the case where a single raw material is used.

### Example 4: Test of Cell Proliferation Promoting Ability of Tetrapeptides

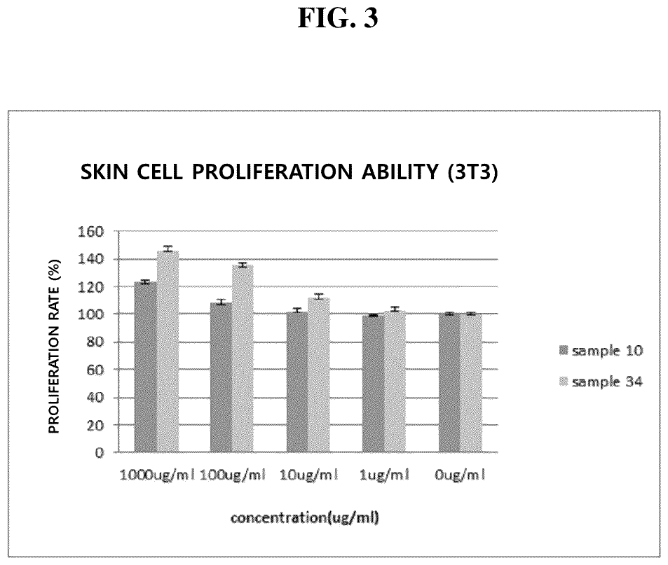

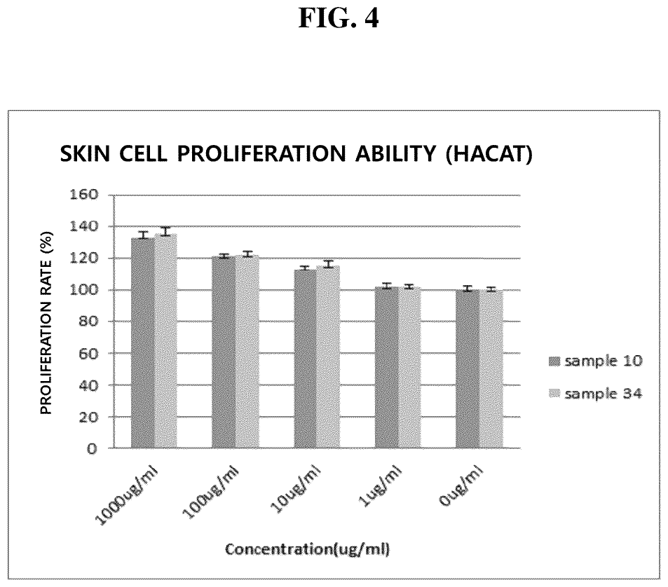

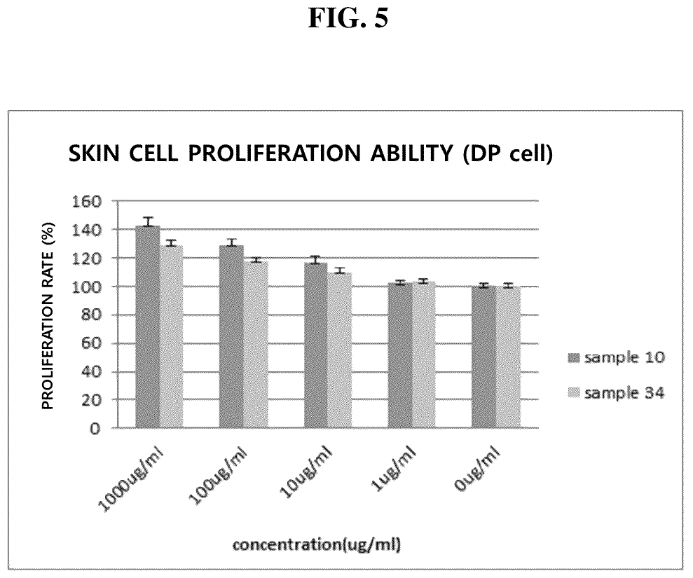

In order to confirm the cell proliferation ability of the two tetrapeptides selected in Examples 2 and 3, an experiment was performed as follows, and the results are shown in FIGS. 3 to 5. In this experiment, the cell proliferation ability was measured using the MTT assay.

3T3 cells, HACAT cells, and DP cells were seeded to a 24-well plate so that each well can have a cell number of 4×10⁴ cells/well, and then incubated at a constant temperature under the conditions of 37° C. and 5% CO₂ for 24 hours. After washing twice with phosphate buffer saline (PBS), the cells were treated with each sample at a concentration of 10 μg/mL and the control group was treated with transforming growth factor-β (TGF-β) a at concentration of 100 ng/mL and incubated at a constant temperature for 24 hours. After the cultivation, 0.5% 3-(4,5-dimethylthiazol-2-yl) -2,5-diphenyltetrazolium bromide (MTT) in Dulbecco's Phosphate-Buffered Saline (DPBS) was mixed with the culture medium at a 1:9 (v/v) ratio and added thereto, and the mixture was cultured in a CO₂ incubator for 2 hours. Thereafter, the generated formazan was dissolved in dimethyl sulfoxide (DMSO), and the absorbance was measured at 570 nm using ELISA.

Referring to FIGS. 3 to 5, with regard to the HACAT cells, 3T3 cells, and DP cells, it was confirmed that the two types of peptides, which were treated from low to high concentrations, have a cell proliferation ability in all of the skin cells in a concentration-dependent manner, and no significant changes were observed in the microscopic observation of these cells.

From the foregoing, it can be seen that the tetrapeptides according to the present invention have no toxicity to skin-related cells, and the tetrapeptides appear to have an effect on skin cell proliferation. Therefore, it can be predicted that the cosmetic composition containing these tetrapeptides according to the present invention will not have any significant side-effect even when it is treated on the skin.

### Example 5: HPLC Analysis of Tetrapeptides

HPLC analysis conditions for accurate analysis and quantification of the two selected tetrapeptides were established.

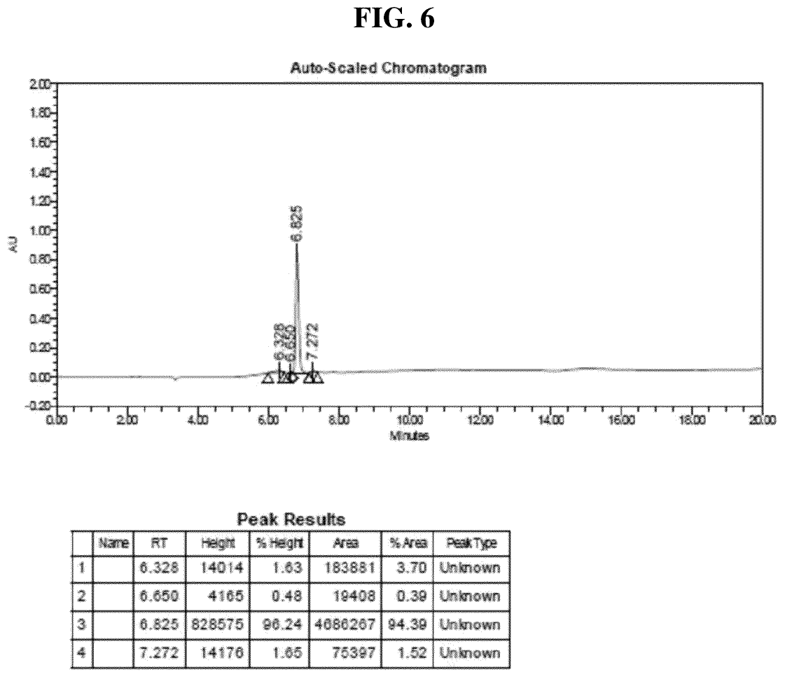

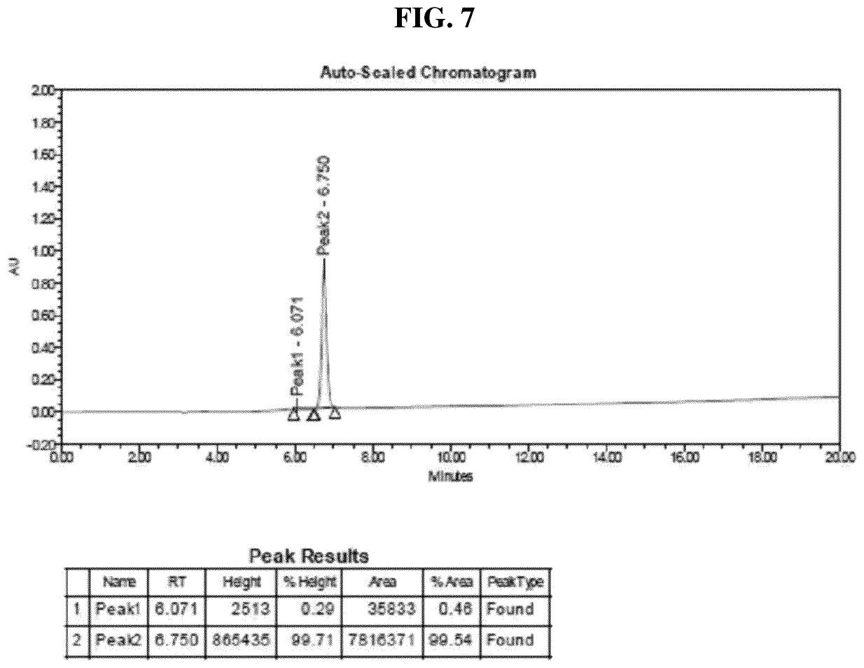

Each peptide was analyzed using the HPLC 2695 (the Waters™) and C18 column (Waters™, Xterra MS C18 column; L: 250 mm, LD: 4.6 mm, 5 μm). In particular, the wavelength of the detector of HPLC used was 216 nm. As solvents for the mobile phase of HPLC, acetonitrile (ACN) containing 0.1% trifluoroacetic acid (TFA) and water containing 0.1% TFA were used. The purity analysis of the peptides was observed by changing the concentration of ACN with time, and the results are shown in FIGS. 6 and 7. The conditions required for the analysis of peptide materials are shown in Table 3 below.

TABLE 3
Conditions of HPLC analysis

| Column | Waters ™ Xterra MS column | | | |
|---|---|---|---|---|
| | (L: 250 mm, LD: 4.6 mm, 5 μm) | | | |
| Detector | Waters ™ 2487 Dual Absorbance Detector | | | |
| Detection wavelength | 216 nm | | | |
| Flow rate | 1.0 mL/min | | | |
| Injection volume | 50 μL | | | |
| Mobile phase | | | 0.1% TFA | 0.1% TFA |
| conditions for HPLC | Program | Time | in DW | in ACN |
| gradient-elution | order | (min) | (%) | (%) |
| | 1 | 0 | 100 | 0 |
| | 2 | 10 | 75 | 25 |
| | 3 | 20 | 50 | 50 |
| | 4 | 30 | 25 | 75 |
| | 5 | 40 | 0 | 100 |
| | 6 | 45 | 100 | 0 |

Referring to FIGS. 6 and 7, as a result of HPLC analysis, it was confirmed that the two types of tetrapeptide could be : accurately identified under the conditions of the HPLC analysis and their accurate concentrations could be known.

### Example 6: Confirmation of Thermal and Photo Stabilities of Tetrapeptides

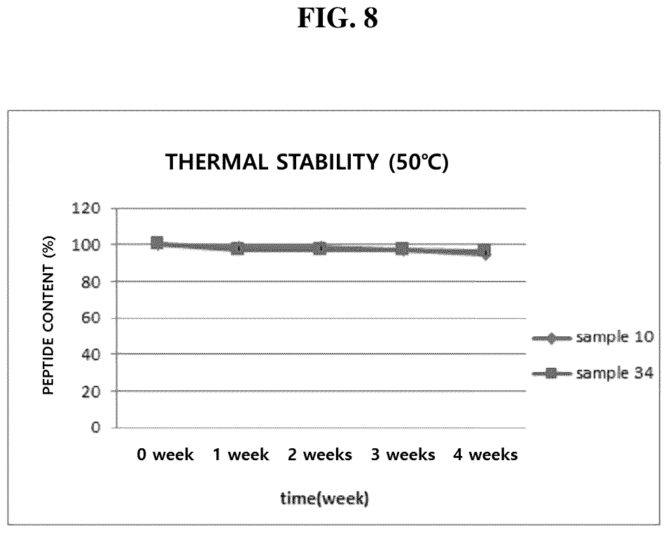

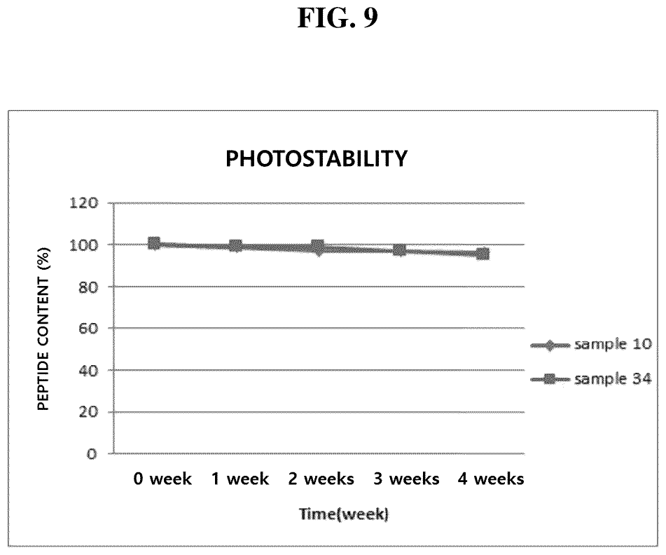

In order to confirm the thermal and photo stabilities of the two types of tetrapeptides, experiments were conducted in the following manner, and the results are shown in FIGS. 8 and 9.

Each sample was dissolved in 50 mM Tris-HCl (pH 8.0) buffer to a concentration of 10 mg/mL and then seeded into a glass vial. Each vial was stored at 50° C. for 4 weeks, and the loss of peptides in the composition due to heat was measured, and stability under sunlight conditions was measured in the same manner.

Referring to FIGS. 9 and 10, as a result of measuring the loss of peptides due to heat and measuring the stability under sunlight conditions, the two tetrapeptides merely showed a peptide loss of less than 10% up to 4 weeks, confirming that these tetrapeptides had high stabilities during the storage period under heat and sunlight.

### Example 7: Preparation of Cosmetic Composition Containing Tetrapeptides

A cream-type composition containing a tetrapeptide was prepared by the following method. As a cream type composition, one part by weight of the tetrapeptide of Sample 10 was included to constitute formulations as shown in Table 4 below.

### 1) Preparation of [Aqueous Phase B]

Powdered raw materials are slowly added in sequence while heating water and stirring at 60° C. to 70° C., stirred with a disper mixer until the mixture becomes thickened and loosened evenly, allowed to cool, and prepared in advance for ease of processing.

### 2) Preparation of [Aqueous Phase A]

After the powdered raw materials are sufficiently impregnated with glycerin, water is added thereto and heated to 50° C. to 60° C. to dissolve the mixture evenly with a disper mixer.

### 3) Preparation by Mixed Stirring of [Aqueous Phase A]+[Aqueous Phase B]

Aqueous phase B prepared in advance is added to aqueous phase A, and the mixture is heated while mixing them the mixture evenly. The mixture is prepared to be maintained at a temperature between 65° C.and 75° C.

### 4) Preparation by Mixed Stirring of [Emulsion Phase]

Each raw material is weighed in an individual beaker and melted by heating and stirring (80° C. or higher). The molten phase is prepared so that the temperature can be maintained at 75° C. or higher.

### 5) Preparation of Whole Cream

The emulsion phase is slowly added to the prepared aqueous phase mixture and stirred vigorously (6,000 rpm) for about 10 minutes to proceed with the emulsification process. In particular, the temperature should be maintained at a temperature between 65° C. and 75° C.

After the completion of the above process, additive A is prepared and the process of pH adjustment and neutralization is performed. In particular, since the viscosity of the contents increases, the resultant is stirred vigorously (6,000 rpm) evenly for 5 minutes or more.

When the contents become cooled down to 50° C. or below, additive B is added thereto and mixed slowly for about 2 minutes (3,000 rpm to 4,000 rpm). When the temperature reaches 40° C. or below, additive C and additive D are added sequentially, and the mixture is stirred slowly (3,000 rpm to 4,000 rpm) for about 2 minutes, and thereafter, the contents are collected through a degassing/filtration process.

After the completion of the above process, the process of pH adjustment and neutralization is performed. In particular, since the viscosity of the contents increases, the resultant is stirred vigorously (6,000 rpm) and evenly for 5 minutes or more.

When the contents become cooled down to 50° C. or below, a preservative is added thereto and mixed slowly for about 2 minutes (3,000 rpm to 4,000 rpm). When the temperature reaches 40° C. or below, a tetrapeptide and a fragrance are added thereto, and the mixture is stirred slowly (3,000 rpm to 4,000 rpm) for 2 minutes, thereafter, the contents are collected through a degassing/filtration process.

The prepared cream was stored under conditions of a low temperature (4° C.), a high temperature (50° C.), room temperature (25° C.), and sunlight to confirm the stability of the cream. No phenomena such as discoloration, an altered odor, a phase change, and a pH change of the cream were observed for 4 weeks under all of the conditions above, confirming that the stability of the cream was maintained. In particular, the stabilities of the tetrapeptides were maintained, and the results of HPLC analysis confirmed that the contents were maintained at 90% or more for 4 weeks under all of the conditions above.

TABLE 4

| | Name of Raw | | Parts |
|---|---|---|---|
| | Materials/Name | Name of Ingredients/ | by |
| Category | of Ingredients | INCI Name | weight |
| Aqueous | DI water | water | 48.04 |
| Phase A | Glycerin | glycerin | 10.00 |
| | Keltrol F | xanthan gum | 0.10 |
| | Adenosine | adenosine | 0.04 |
| Aqueous | DI water | water | 30.00 |
| Phase B | Beta-Glucan | beta-glucan | 0.20 |
| | Carbopol 940 | carbomer | 0.02 |
| Emulsion | Kalcol 6870 | cetearyl alcohol | 2.50 |
| Phase | Gms-105 | glyceryl stearate | 1.50 |
| | Arlacel 165V | glyceryl stearate, peg- | 1.20 |
| | | 100 stearate | |
| | Tween 80 | polysorbate 80 | 1.00 |
| | Arlacel 83 | sorbitan sesquioleate | 0.70 |
| | Phytosqualane | squalane | 5.00 |
| | Dub Mct | caprylic/capric | 3.00 |
| | | triglyceride | |
| Additive | DI Water | water | 3.00 |
| A | L-Arginine (pH | arginine | 0.50 |
| | 4.50 to pH 5.50) | | |
| Additive | 1, 2-Hexanediol | 1, 2-hexanediol | 2.00 |
| B | | | |
| Additive | Lutein Solution | lutein | 1.00 |
| C | | | |
| Additive | Perfume | fragrance | 0.10 |
| D | | | |
| (fragrance) | | | |
| Peptide | GQVS | peptide | 0.1 |
| to be | (SEQ ID NO: 1) | | |
| added | | | |
| later | | | |

Heretofore, preferred embodiments of the present invention have been described in detail with reference to the accompanying drawings. The description of the present invention is illustrative and those skilled in the art could understand that modification into other specific forms could easily be made without changing the spirit and essential features of the present invention.

Thus, the scope of the present invention is represented not by the description of the present invention but by claims to be set forth later, and it should be understood that all the modified or changed forms derived from the meaning, scope, and equivalents thereto are included in the scope of the present invention.

What is claimed is:

1. A method of synthesizing the tetrapeptide exhibiting an anti-wrinkle activity consisting of the amino acid sequence of glycine(Gly)-glutamine(Gln)-valine(Val)-serine(Ser) (SEQ ID NO: 1), the method comprising:
preparing a peptidyl resin by utilizing 2-(4-nitrophenyl) sulfonylethoxycarbonyl-amino acid (Nsc-amino acid);
adding an eluting solution to the prepared resin; and
filtering the resin and obtaining the tetrapeptide.


SEQUENCE LISTING (42 sequences)

SEQ ID NO: 1 - protein, 4 aa, synthetic construct
  REGION 1..4: note = Tetra Peptide
GQVS

SEQ ID NO: 2 - protein, 4 aa, synthetic construct
  REGION 1..4: note = Tetra Peptide
GQDP

SEQ ID NO: 3 - protein, 4 aa, synthetic construct
  REGION 1..4: note = Tetra Peptide
MSYQ

SEQ ID NO: 4 - protein, 4 aa, synthetic construct
  REGION 1..4: note = Tetra Peptide
QKKQ

SEQ ID NO: 5 - protein, 4 aa, synthetic construct
  REGION 1..4: note = Tetra Peptide
PTPQ

SEQ ID NO: 6 - protein, 4 aa, synthetic construct
  REGION 1..4: note = Tetra Peptide
PPVD

SEQ ID NO: 7 - protein, 4 aa, synthetic construct
  REGION 1..4: note = Tetra Peptide
DCVK

SEQ ID NO: 8 - protein, 4 aa, synthetic construct
  REGION 1..4: note = Tetra Peptide
VKTS

SEQ ID NO: 9 - protein, 4 aa, synthetic construct
  REGION 1..4: note = Tetra Peptide
KTSG

SEQ ID NO: 10 - protein, 4 aa, synthetic construct
  REGION 1..4: note = Tetra Peptide
SGGS

SEQ ID NO: 11 - protein, 4 aa, synthetic construct
  REGION 1..4: note = Tetra Peptide
GSGY

SEQ ID NO: 12 - protein, 4 aa, synthetic construct
  REGION 1..4: note = Tetra Peptide
GYVS

SEQ ID NO: 13 - protein, 4 aa, synthetic construct
  REGION 1..4: note = Tetra Peptide
SQQV

SEQ ID NO: 14 - protein, 4 aa, synthetic construct
  REGION 1..4: note = Tetra Peptide
TQTS

SEQ ID NO: 15 - protein, 4 aa, synthetic construct
  REGION 1..4: note = Tetra Peptide
CAPQ

SEQ ID NO: 16 - protein, 4 aa, synthetic construct
  REGION 1..4: note = Tetra Peptide
QPSY

SEQ ID NO: 17 - protein, 4 aa, synthetic construct
  REGION 1..4: note = Tetra Peptide
YGGG

SEQ ID NO: 18 - protein, 4 aa, synthetic construct
  REGION 1..4: note = Tetra Peptide
SSGG

SEQ ID NO: 19 - protein, 4 aa, synthetic construct
  REGION 1..4: note = Tetra Peptide
GGSG

SEQ ID NO: 20 - protein, 4 aa, synthetic construct
  REGION 1..4: note = Tetra Peptide
GSSG

SEQ ID NO: 21 - protein, 4 aa, synthetic construct
  REGION 1..4: note = Tetra Peptide
GCFS

SEQ ID NO: 22 - protein, 4 aa, synthetic construct
  REGION 1..4: note = Tetra Peptide
CFSS

SEQ ID NO: 23 - protein, 4 aa, synthetic construct
  REGION 1..4: note = Tetra Peptide
FSSG

SEQ ID NO: 24 - protein, 4 aa, synthetic construct
  REGION 1..4: note = Tetra Peptide
GQLE

SEQ ID NO: 25 - protein, 4 aa, synthetic construct
  REGION 1..4: note = Tetra Peptide
ELPE

SEQ ID NO: 26 - protein, 4 aa, synthetic construct
  REGION 1..4: note = Tetra Peptide
EQQE

SEQ ID NO: 27 - protein, 4 aa, synthetic construct
  REGION 1..4: note = Tetra Peptide
LELP

SEQ ID NO: 28 - protein, 4 aa, synthetic construct
  REGION 1..4: note = Tetra Peptide
GQLK

SEQ ID NO: 29 - protein, 4 aa, synthetic construct
  REGION 1..4: note = Tetra Peptide
KHLE

SEQ ID NO: 30 - protein, 4 aa, synthetic construct
  REGION 1..4: note = Tetra Peptide
EHQE

SEQ ID NO: 31 - protein, 4 aa, synthetic construct
  REGION 1..4: note = Tetra Peptide
EGQL

SEQ ID NO: 32 - protein, 4 aa, synthetic construct
  REGION 1..4: note = Tetra Peptide
LEVP

SEQ ID NO: 33 - protein, 4 aa, synthetic construct
  REGION 1..4: note = Tetra Peptide
PEEQ

SEQ ID NO: 34 - protein, 4 aa, synthetic construct
  REGION 1..4: note = Tetra Peptide
QMGQ

SEQ ID NO: 35 - protein, 4 aa, synthetic construct
  REGION 1..4: note = Tetra Peptide
QLKY

SEQ ID NO: 36 - protein, 4 aa, synthetic construct
  REGION 1..4: note = Tetra Peptide
YLEQ

SEQ ID NO: 37 - protein, 4 aa, synthetic construct
  REGION 1..4: note = Tetra Peptide
EQQE

SEQ ID NO: 38 - protein, 4 aa, synthetic construct
  REGION 1..4: note = Tetra Peptide
TKGE

SEQ ID NO: 39 - protein, 4 aa, synthetic construct
  REGION 1..4: note = Tetra Peptide
VLLP

SEQ ID NO: 40 - protein, 4 aa, synthetic construct
  REGION 1..4: note = Tetra Peptide
VEHQ

SEQ ID NO: 41 - protein, 4 aa, synthetic construct
  REGION 1..4: note = Tetra Peptide
QQKQ

SEQ ID NO: 42 - protein, 4 aa, synthetic construct
  REGION 1..4: note = Tetra Peptide
EVQW